(19) United States
(12) Patent Application Publication
(10) Pub. No.: US 2014/0212875 A1
(43) Pub. Date: Jul. 31, 2014

(54) Human Metabolically Active Brown Adipose Derived Stem Cells

(71) Applicant: Francisco Javier Silva, Miramar, FL (US)

(72) Inventor: Francisco Javier Silva, Miramar, FL (US)

(73) Assignee: BioRestorative Therapies, Inc., Jupiter, FL (US)

(21) Appl. No.: 14/163,594

(22) Filed: Jan. 24, 2014

Related U.S. Application Data
(60) Provisional application No. 61/756,857, filed on Jan. 25, 2013.
(60) Provisional application No. 61/757,900, filed on Jan. 29, 2013.

(51) Int. Cl. C12Q 1/68

(52) U.S. Cl. CPC C12Q 1/6881
USPC 435/6.11

(57) ABSTRACT

A method of distinguishing a brown adipose cell from a white adipose cell. In one embodiment the method includes measuring the expression level of one or more genes in an adipose cell; comparing the measured expression levels to a control, and correlating the expression level of the one or more genes to an identity as a white adipose cell or a brown adipose cell. In one embodiment the one or more genes are selected from the genes listed in FIG. 4C. In another aspect the invention relates to a method of differentiating an adipose stem cell. In one embodiment the method includes inducing differentiation of an adipose stem cell in vitro; and distinguishing the differentiated stem cell. In another embodiment the inducing is performed by contacting the adipose stem cell with a brown adipose cell differentiation media.

### RELATED APPLICATIONS

This application claims priority to U.S. Provisional applications 61/756,857 filed Jan. 25, 2013 and 61/757,900 filed Jan. 29, 2013; the contents of each are herein incorporated in their entirety.

### FIELD OF THE INVENTION

The invention relates generally to the field of cell culture and more specifically to the field of determining cell type.

### BACKGROUND

Brown adipose tissue (BAT) plays a key role in the evolutionarily conserved mechanisms underlying energy homeostasis in mammals. It is characterized by fat vacuoles 5-10 microns in diameter and expression of uncoupling protein 1 (UCP1), central to the regulation of thermogenesis. In the human newborn, depots of BAT are typically grouped around the vasculature and solid organs. These depots maintain body temperature during cold exposure by warming the blood before its distribution to the periphery. They also ensure an optimal temperature for biochemical reactions within solid organs. BAT had been thought to involute throughout childhood and adolescence. Recent studies, however, have confirmed the presence of active brown adipose tissue in adult humans with depots residing in cervical, supraclavicular, mediastinal, paravertebral and suprarenal regions. While human pluripotent stem cells have been differentiated into functional brown adipocytes in vitro and inducible brown adipocyte progenitor cells have been identified in murine skeletal muscle and white adipose tissue, metabolically active brown adipose tissue derived stem cells have not been identified in adult humans to date.

The present invention addresses this issue.

### SUMMARY OF THE INVENTION

In one aspect the invention relates to a method of distinguishing a brown adipose cell from a white adipose cell. In one embodiment the method includes measuring the expression level of one or more genes in an adipose cell; comparing the measured expression levels to a control, and correlating the expression level of the one or more genes to an identity as a white adipose cell or a brown adipose cell. In one embodiment the one or more genes are selected from the genes listed in FIG. 4C. In another embodiment an increase in expression of one or more of the following genes as compared to the control is indicative that the adipose cell is a brown adipose cell: ACACB, ADRB2, FGF10, KLF15, LIPE, NR1H3, CIDEC, ELOVL3, INHBB, PPARGC1A, and UCP1. In yet another embodiment an increase in expression of LEP as compared to the control is indicative that the adipose cell is a white adipose cell. In still yet another embodiment the method measures the expression level by quantifying transcript levels.

In one embodiment the method measures the levels of at least two, at least three, at least four, at least five, at least 6, at least 7, at least 8, at least 9, at least 10, or at least 11 genes. In another embodiment the method measures the levels of any one of ELOVL3, INHBB, PPARGC1A, or UCP1. In yet another embodiment the method measures the levels of any two of ELOVL3, INHBB, PPARGC1A, or UCP1. In still yet another embodiment the method measures the levels of any three of ELOVL3, INHBB, PPARGC1A, or UCP1. In one embodiment the method measures the levels of ELOVL3, INHBB, PPARGC1A, and UCP1.

In another aspect the invention relates to a method of differentiating an adipose stem cell. In one embodiment the method includes inducing differentiation of an adipose stem cell in vitro; and distinguishing the differentiated stem cell. In another embodiment the inducing is performed by contacting the adipose stem cell with a brown adipose cell differentiation media. In yet another embodiment the inducing is performed by contacting the adipose stem cell with FNDC5.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The present teachings described herein will be more fully understood from the following description of various illustrative embodiments, when read together with the accompanying drawings. It should be understood that the drawings described below are for illustration purposes only and are not intended to limit the scope of the present teachings in anyway.

FIG. 1(A) is a series of diagrams of flow cytometry results of undifferentiated brown adipose derived stem cells;

FIG. 1(B) is a photomicrograph of biopsied mediastinal brown adipose depots that demonstrate multiocular lipid morphology and UCP1 staining specific to brown adipose tissue;

FIG. 1(C) is a karyotype analysis of passage 10 brown adipose derived stem cells;

FIG. 2 is a set of flow cytometry results of TMEM26 and CD137 of brown and white adipose derived stem cells;

FIG. 3(A) is a Western blot of cells 21 days post FNDC5 induction;

FIG. 3(B) is a photomicrograph of Alcian blue stained brown adipose derived stem cells directionally differentiated into chondrocytes;

FIG. 3(C) is a photomicrograph of fatty acid binding protein 4 (FABP4) immunocytochemistry of brown adipose derived stem cells induced to undergo white adipogenesis;

FIG. 3(D) is a photomicrograph of Alizarian red stained brown adipose derived stem cells induced to undergo osteogenesis;

FIG. 4(A) is a SEM of brown adipose derived stem cells cultured on porous extracellular matrix scaffolds;

FIG. 4(B) is a SEM of directionally differentiated brown adipocytes on scaffolds;

FIG. 4(C) is a transcriptional profile of brown adipose derived stem cells differentiated into brown and white adipocytes;

FIG. 4(D) is a measure of fatty acid uptake of brown fat differentiated brown adipose derived stem cells at 7, 14 and 21 days post differentiation;

FIG. 4(E) is a measure of functional mitochondrial respiration assay of brown adipose derived stem cells differentiated into brown adipocytes at 7, 14 and 21 days post differentiation;

FIG. 5(A) is a graph of expression levels of MSC associated genes;

FIG. 5(B) is a graph of expression levels of MSC specific genes; and

FIG. 5(C) is a graph of expression levels of sternness genes.

### DETAILED DESCRIPTION

Briefly, for this study, human adipose tissues were biopsied and analyzed with immunohistochemistry and primary cell isolation. Primary cells isolated from adipose explants were expanded and their growth kinetics, karyotyping, flow cytometry and immunocytochemistry were determined. Passage-2 cells were directionally differentiated into osteogenic, chondrogenic, white adipogenic and brown adipogenic lineages on plastic and also differentiated into brown adipocytes on porous extracellular matrix scaffolds. Differentiation was confirmed by Western blot, immunohistochemistry, cytochemistry, scanning electron microscopy (SEM), and quantitative real-time PCR. Functional brown fat differentiation was confirmed by fatty acid uptake and mitochondrial respiration, as measured by the oxygen consumption rate (OCR).

### Methods

### Mediastinal Adipose Tissue Procurement

Mediastinal adipose tissues were obtained from 54 patients undergoing cardiac surgery. The group included 44 males and 10 females and had a mean ±SE age 72.4±12 yr. (range 28-84 yr.).

### Derivation of Mediastinal Adipose Derived MSCs

The excised tissue was cut into 3 mm pieces and explanted onto a 6 well dish and grown in DMEM low glucose, 10% XcytePL™ Supplement (JadiCell, Phoenix, Ariz.), 1X Glutamax, and 1X MEM-NEAA (Life Technologies, Carlsbad, Calif.) and cultured in 5% CO₂/37° C.

### RNA Analysis

RNA was isolated and DNaseI treated using the RNAqueous-4PCR Kit (Life Technologies AM1914 (Life Technologies, Carlsbad, Calif.)) per manufacturer's protocol.

First strand cDNA was synthesized using the RI' First Strand Kit (SABiosciences 330401) (SABiosciences, Valencia, Calif.) per manufacturer's protocol.

PCR was carried out on RT² Profiler PCR Arrays using RT²SYBR Green qPCR Mastermix (SABiosciences 330521) in an Eppendorf Mastercycler ep realplex 4 pcr machine (Eppendorf, Hauppauge, N.Y.) per manufacturer's protocol.

The following RT² Profiler PCR Arrays and individual gene primers were used:
Human Adipogenesis (SABiosciences PAHS-049A)
Human Mesenchymal Stem Cells (SABiosciences PAHS-082A)
RT² qPCR Primer Assay for CIDEC(SABiosciences PPH18299E)
RT² qPCR Primer Assay for COX8A (SABiosciences PPH20233A)
RT² qPCR Primer Assay for CYC1(SABiosciences PPH00724A)
RT² qPCR Primer Assay for CYFIP2 (SABiosciences PPH14474E)
RT² qPCR Primer Assay for DPT (SABiosciences PPH10191A)
RT² qPCR Primer Assay for ELOVL3(SABiosciences PPH16532A)
RT² qPCR Primer Assay for INHBB(SABiosciences PPH01917A)
RT² qPCR Primer Assay for LHX8(SABiosciences PPH19135A)
RT² qPCR Primer Assay for NDUFA11(SABiosciences PPH19207A)
RT² qPCR Primer Assay for NDUFA13(SABiosciences PPH60028A)
RT² qPCR Primer Assay for PMP22(SABiosciences PPH02152E)
RT² qPCR Primer Assay for GJA1(SABiosciences PPH02781E)
RT² qPCR Primer Assay for MYH7(SABiosciences PPH00044E)
RT² qPCR Primer Assay for NKX2-5(SABiosciences PPH02462A)
RT² qPCR Primer Assay for TNNT2(SABiosciences PPH02619A)
RT² qPCR Primer Assay for B2M(SABiosciences PPH01094E)
RT² qPCR Primer Assay for HPRT1(SABiosciences PPH01018B)
RT² qPCR Primer Assay for RPL13A(SABiosciences PPH01020B)

Delta delta (ΔA) Ct based fold-change calculations were performed using the RT² Profiler PCR Array Data Analysis Web Portal version 3.5 provided by SABiosciences at: http://pcrdataanalysis.sabiosciences.com/pcr/arrayanalysis.php

Total RNA was purified and DNase-treated from individual wells of 24-well plates using the RNAqueous-Micro kit (Ambion AM1931(Life Technologies, Carlsbad, Calif.)) per manufacturer's protocol. 100 ng of each sample was reverse transcribed and pre-amplified using the RT² PreAMP cDNA Synthesis Kit (SABiosciences 330241) (SABiosciences, Valencia, Calif.). The preamplified product was then amplified using the RT² SYBR Green/ROX qPCR Master Mix (SABiosciences PA-012) (SABiosciences, Valencia, Calif.). Experiments were done in triplicate and data was analysed by the delta delta (ΔA) Ct method. The control gene used was HPRT1.

### Immunocytochemistry

Cells were fixed with 4% paraformaldehyde. After blocking, cells were incubated with primary antibody diluted in 5% donkey serum. After washing cells were incubated with secondary antibody and counterstained with DAPI (Molecular Probes (Life Technologies, Carlsbad, Calif.)). For negative controls, incubation without primary antibody and with corresponding specific non-immune immunoglobulin (EMD Millipore, Billerica, Mass.) was used.

### Flow Cytometry

Directly conjugated antibodies used: HLA-DP DQ DR (BD Biosciences,), CD90, LIN, CD166, STRO-1, SSEA-4, CD44, CD106, CD73, CD117, CD105, HLA-ABC, CD86, CD63, CD9, CD80 (Biolegend, San Diego, Calif.), CD45, CD133, and CD34 (Miltenyi Biotech, Bergisch Gladbach, Germany). After staining, the cells were fixed and analyzed using a FACSCanto II analyzer (BD Biosciences, Franklin Lakes, N.J.).

### Adipogenic Differentiation

Cells were plated in 6-well dishes at a density of 50,000 cells/well. White or brown adipogenesis differentiation medium was added. For brown adipogenesis, FNDC5 was added 6 days post induction. Fatty acid binding protein 4 (FABP4) immunocytochemistry and 0.3% Oil Red O (Sigma Aldrich, St. Louis, Mo.) was used for staining to detect intracellular lipid accumulation (Data not shown).

### Osteogenic Differentiation

Cells were plated in 6 well dishes at a density of 50,000 cells/well. StemPro® Osteogenesis Differentiation medium ((Life Technologies, Carlsbad, Calif.)) was added. 2% Alizarian Red S (Sigma Aldrich, St. Louis, Mo.) was used for staining to detect de novo formation of bone matrix.

### Chondrogenic Differentiation

500,000 cells/15 ml tube were pelleted and induced with StemPro® Chondrogenesis Differentiation medium ((Life Technologies, Carlsbad, Calif.)). 1% Alcian Blue (Sigma Aldrich, St. Louis, Mo.) was used to detect sulfated glycosaminoglycans.

### Fatty Acid Uptake Analysis

Analysis began with the replacement of growth media with HBSS Buffer with 20 mM HEPES and 0.2% fatty free BSA. Cells were placed in the incubator for 1.5 h, QBT Fatty Acid Uptake (Molecular Devices, Sunnyvale, Calif.) media was added to the wells and fluorescence was analyzed every minute in a Bio-Tek Synergy HT (Bio Tek, Winooski, Vt.).

### Cellular Respiration and Glycolysis Analysis

The oxygen consumption rate (OCR) was performed using a Seahorse Bioscience XF-24 instrument (Seahorse Bioscience, Billerica, Mass.). Analysis was performed by replacing the growth media with XF assay media and incubating in a CO₂ free chamber for 1 h. The XF Cell Mito Stress Test simultaneously analyzed basal respiration, ATP turnover, proton leak, spare respiratory capacity and glycolysis.

### Transmission Electron Microscopy

Samples were fixed and embedded for routine TEM. They were then examined on an FEI Tecnai T-12 (FEI Hillsboro, Oreg.) at 120 KV.

### Scanning Electron Microscopy

Scaffolds were fixed and post fixed in 2% osmium tetroxide, dehydrated through a series of ethanol washes, dried with hexamethyldisilazane. Scaffolds were then sputter coated with gold and imaged with a scanning electron microscope under high vacuum.

### Results

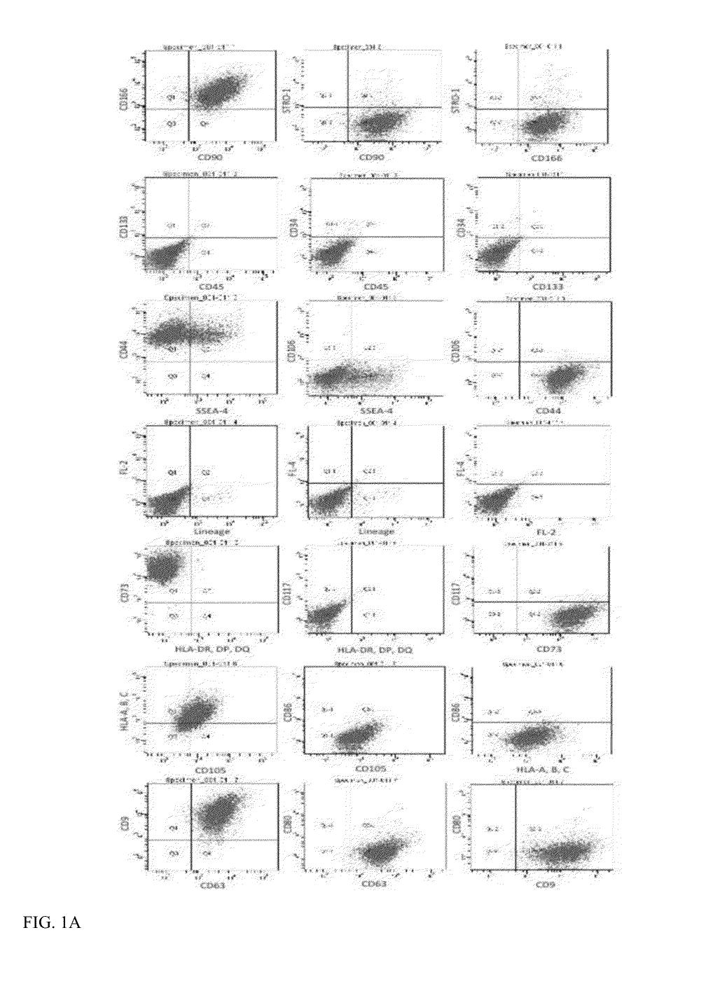

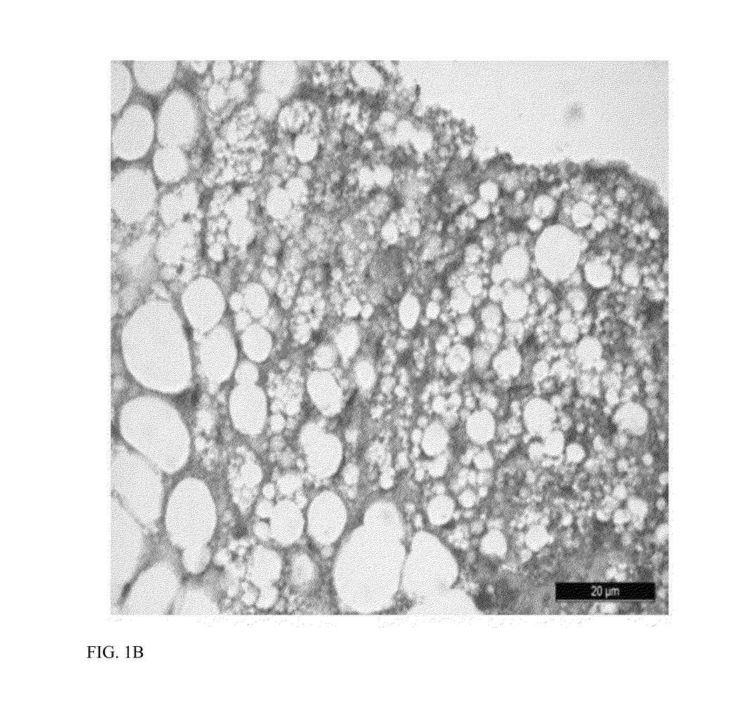

FIG. 1(A) shows flow cytometry of undifferentiated brown adipose derived stem cells. The cells expressed CD44, CD105, CD166, and CD90 and were negative for hematopoietic markers CD34, CD45, and HLA-DR. FIG. 1(B) is a photomicrograph of biopsied mediastinal brown adipose depots demonstrate multiocular lipid morphology and UCP1 staining specific to brown adipose tissue. FIG. 1(C) is a karyotype analysis of passage 10 brown adipose derived stem cells.

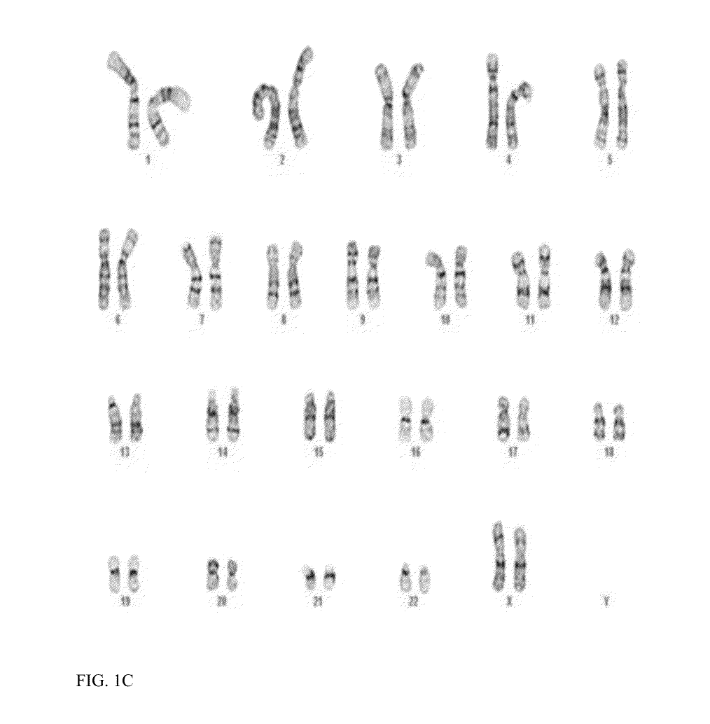

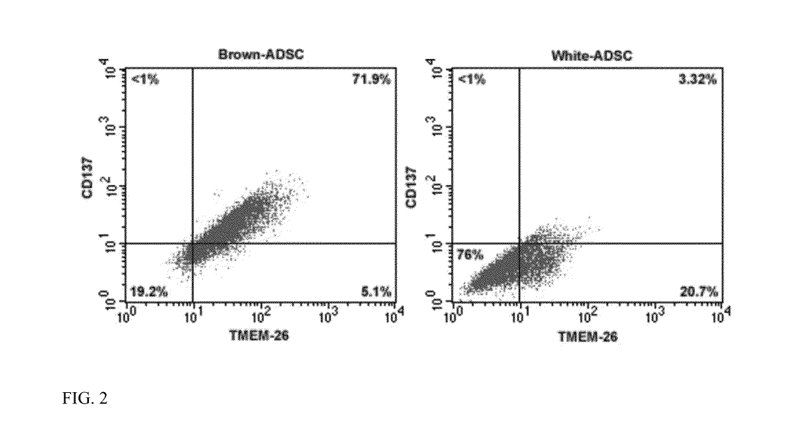

FIG. 2 depicts the flow cytometry results of TMEM26 and CD 137 of brown and white adipose derived stem cells. Brown adipose derived stem cells express higher levels of TMEM26 and CD137.

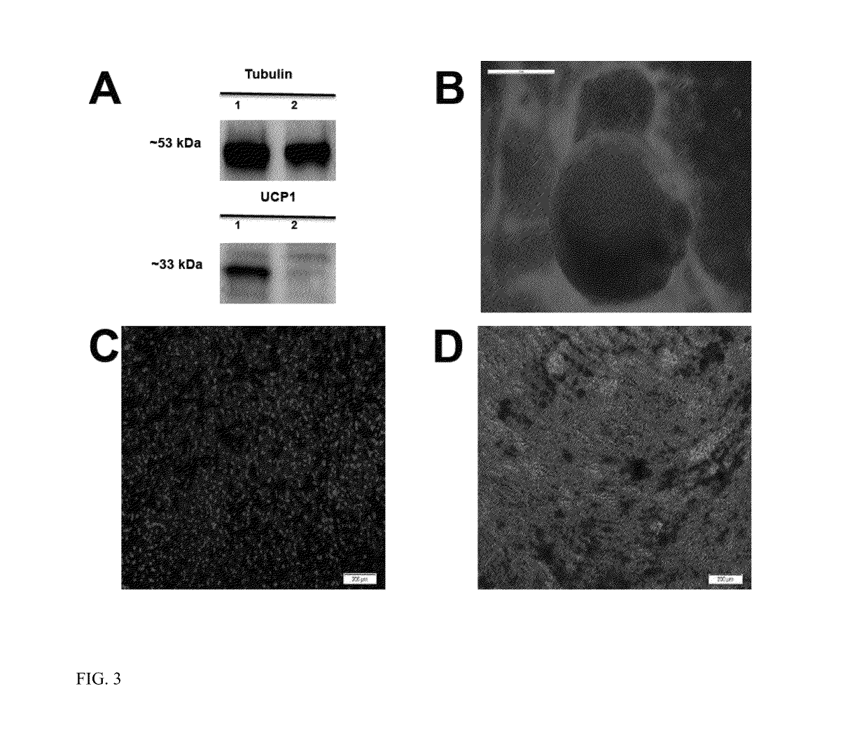

FIG. 3(A) is a Western blot 21 days post FNDC5 induction. Lane 1 holds brown adipose derived stem cells directionally differentiated into brown adipocytes. Lane 2 holds undifferentiated brown adipose derived stem cells. FIG. 3(B) is a photomicrograph of Alcian blue stained brown adipose derived stem cells directionally differentiated into chondrocytes. FIG. 3(C) is a photomicrograph of the fatty acid binding protein 4 (FABP4) immunocytochemistry of brown adipose derived stem cells induced to undergo white adipogenesis. FIG. 3(D) is a photomicrograph of Alizarin red stained brown adipose derived stem cells induced to undergo osteogenesis.

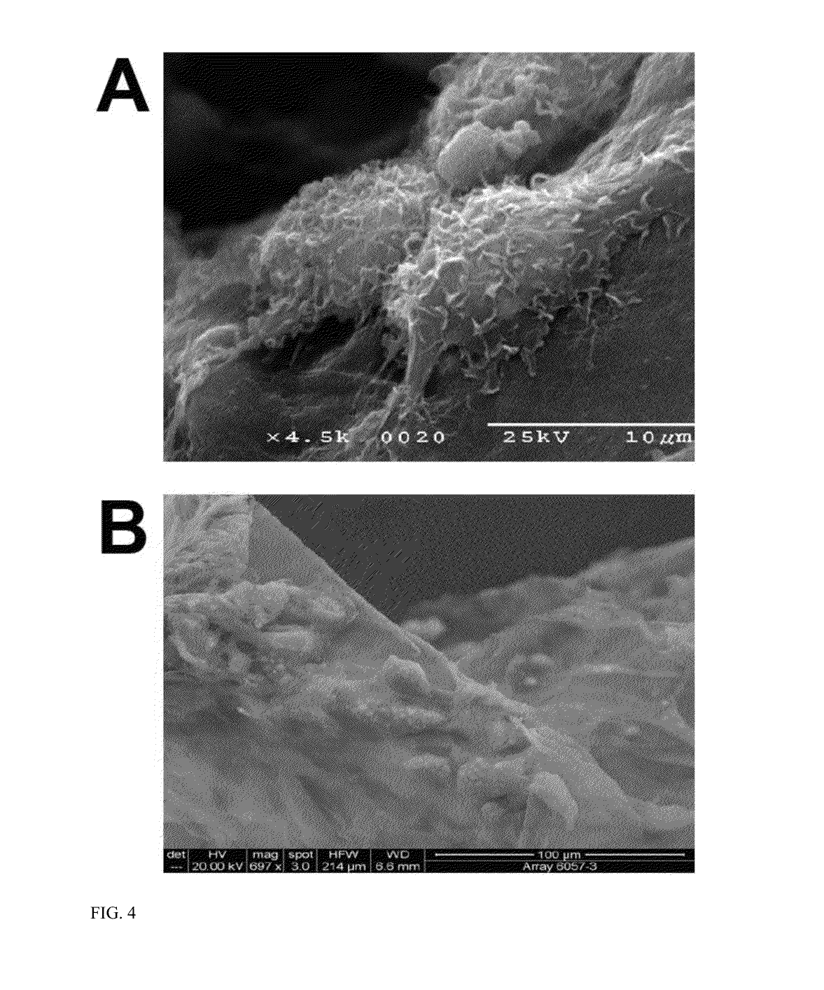

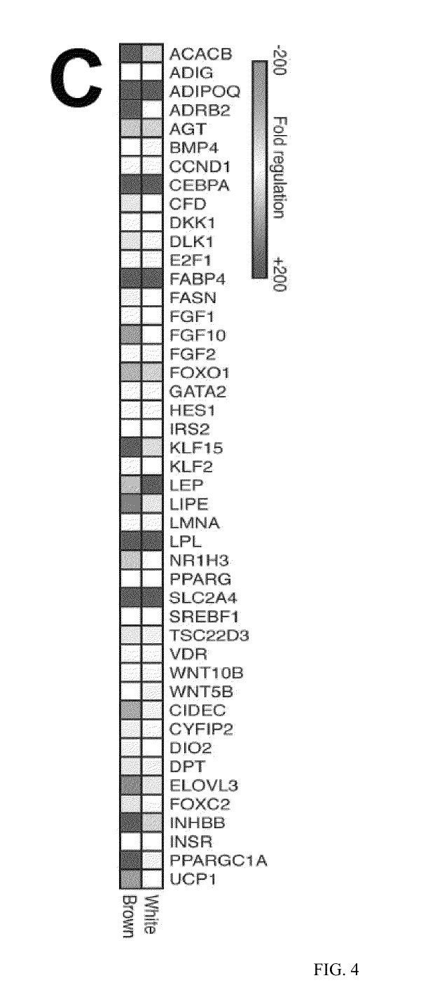

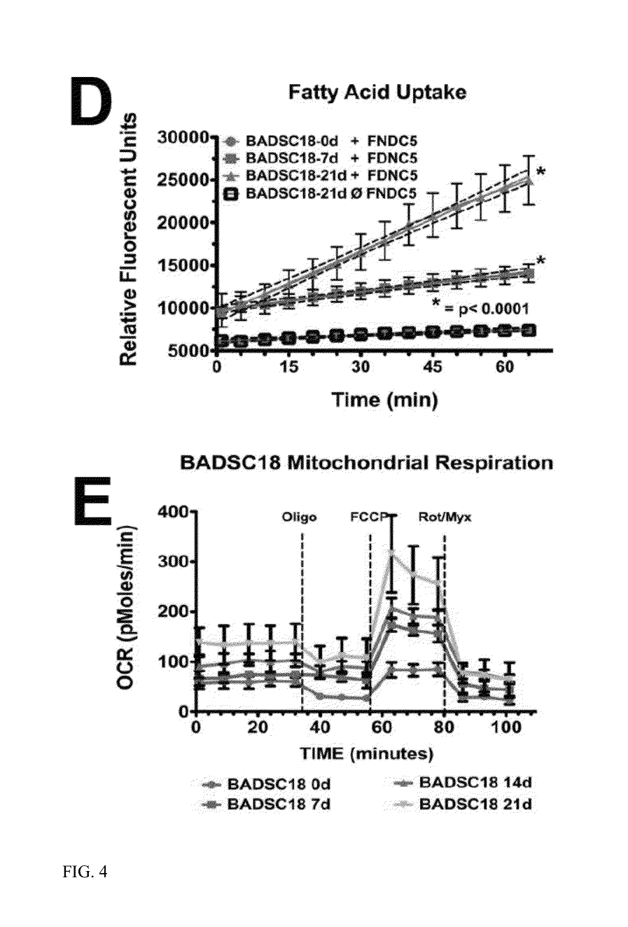

FIG. 4(A) is a SEM of brown adipose derived stem cells cultured on porous extracellular matrix scaffolds. FIG. 4(B) SEM of directionally differentiated brown adipocytes on scaffolds. FIG. 4(C) is a transcriptional profile of brown adipose derived stem cells differentiated into brown and white adipocytes. FIG. 4(D) is a graph of fatty acid uptake of brown fat differentiated brown adipose derived stem cells at 7, 14 and 21 days post differentiation. FIG. 4(E) is a graph of the results of a functional mitochondrial respiration assay of brown adipose derived stem cells differentiated into brown adipocytes at 7, 14 and 21 days post differentiation.

In FIG. 4(C), profiled genes are listed according to their standard abbreviation (NCBI gene profile):
ACACB: acetyl-CoA carboxylase beta
ADIG: adipogenin
ADIPOQ: Adiponectin
ADRB2: adrenoceptor beta 2, surface
AGT: angiotensinogen
BMP4: bone morphogenetic protein 4
CCND1: cyclin D1
CEBPA: CCAAT/enhancer binding protein (C/EBP), alpha
CFD: complement factor D (adipsin)
DKK1: Dickkopf1
DLK1: delta-like 1 homolog
E2F1: E2F transcription factor 1
FABP4: fatty acid binding protein 4
FASN: Fatty acid synthase
FGF1: fibroblast growth factor 1
FGF10: fibroblast growth factor 10
FGF2: fibroblast growth factor 2
FOXO1: forkhead box O1
GATA2: GATA binding protein 2
HES1: hairy and enhancer of split 1
IRS2: insulin receptor substrate 2
KLF15: Kruppel-like factor 15
KLF2: Kruppel-like factor 2
LEP: Leptin
LIPE: hormone-sensitive lipase
LMNA: lamin A/C
LPL: lipoprotein lipase
NR1H3: nuclear receptor subfamily 1, group H, member 3
PPARG: peroxisome proliferative activated receptor, gamma
SLC2A4: solute carrier family 2 (facilitated glucose transporter)
SREBF1: sterol regulatory element binding transcription factor 1
TSC22D3: TSC22 domain family, member 3
VDR: Vitamin D3 receptor
WNT10B: wingless-type MMTV integration site family, member 10B
WNT5B: wingless-type MMTV integration site family, member 5b
CIDEC: cell death-inducing DFFA-like effector c
CYFIP2: Cytoplasmic FMR1-interacting protein 2
DIO2: deiodinase, iodothyronine, type II
DPT: Dermatopontin
ELOVL3: Elongation of very long chain fatty acids protein 3
FOXC2: forkhead box C2
INHBB: inhibin, beta B
INSR: insulin receptor
PPARGC1A: peroxisome proliferative activated receptor, gamma, coactivator 1
UCP1: uncoupling protein 1.

Table 1 is a list of genes expressed by brown and white MSC as measured against a standard along with a measure of their expression relative to the standard. Thus for example The expression of the gene ANXA5 is 1.178 fold higher in brown than in the standard.

TABLE 1

| Gene | Brown | White |
|---|---|---|
| ANXA5 | 1.178 | 0.150 |
| BDNF | 2.573 | 0.555 |
| BGLAP | 1.214 | 0.180 |
| BMP7 | −1.192 | 0.421 |
| COL1A1 | −1.206 | 0.203 |
| CSF2 | −1.019 | 0.315 |
| CSF3 | −1.143 | 0.523 |
| CTNNB1 | 1.243 | 0.190 |
| EGF | 1.240 | 0.510 |
| FUT1 | 5.110 | 2.330 |
| GTF3A | 1.275 | 0.195 |
| HGF | 1.613 | 0.295 |
| ICAM1 | −3.211 | 0.360 |
| IFNG | −1.059 | 0.239 |
| IGF1 | −2.822 | 3.061 |
| IL10 | −1.982 | 4.268 |
| IL1B | 4.532 | 0.720 |
| IL6 | 13.056 | 2.485 |
| ITGB1 | 1.200 | 0.190 |
| KITLG | −1.248 | 0.290 |
| MITF | 1.050 | 0.220 |
| MMP2 | −1.709 | 0.252 |
| NES | 2.346 | 0.455 |
| NUDT6 | 1.643 | 0.120 |
| PIGS | 1.067 | 0.095 |
| PTPRC | 1.347 | 0.805 |
| SLC17A5 | 1.257 | 0.215 |
| TGFB3 | −1.125 | 0.141 |
| TNF | 1.729 | 0.970 |
| VEGFA | 1.502 | 0.320 |
| VIM | −1.347 | 0.292 |
| VWF | −1.382 | 0.891 |
| ALCAM | 4.327 | 0.665 |
| ANPEP | −1.228 | 0.125 |
| BMP2 | 1.035 | 0.125 |
| CASP3 | 1.879 | 0.150 |
| CD44 | 1.866 | 0.305 |
| ENG | 1.454 | 0.145 |
| ERBB2 | 1.228 | 0.250 |
| FUT4 | 1.272 | 0.205 |
| FZD9 | 1.329 | 0.650 |
| ITGA6 | 12.524 | 2.395 |
| ITGAV | 1.206 | 0.165 |
| KDR | 14.254 | 5.895 |
| MCAM | 1.431 | 0.260 |
| NGFR | −1.102 | 0.440 |
| NT5E | 1.725 | 0.225 |
| PDGFRB | −2.139 | 0.100 |
| PROM1 | 0.010 | 0.000 |
| THY1 | −1.082 | 0.115 |
| VCAM1 | 15.780 | 9.470 |
| FGF2 | 1.643 | 0.275 |
| INS | 0.000 | 0.000 |
| LIF | 3.855 | 0.805 |
| POU5F1 | 3.767 | 0.925 |
| SOX2 | 0.000 | 0.000 |
| TERT | 0.000 | 0.000 |
| WNT3A | 0.000 | 0.000 |
| ZFP42 | 2.240 | 0.670 |

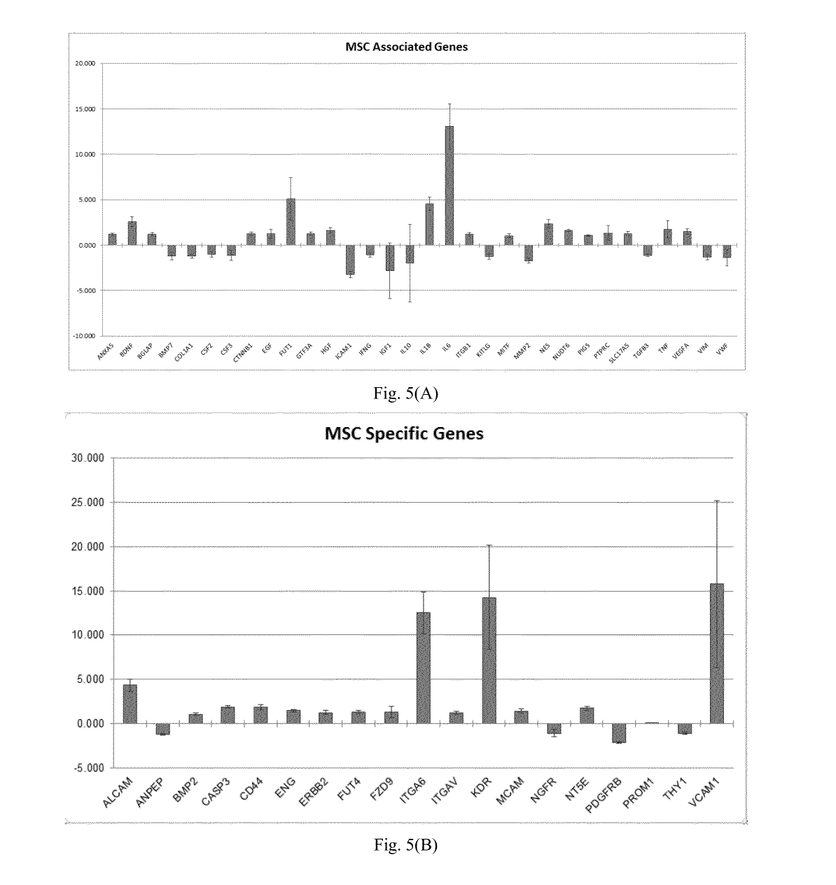

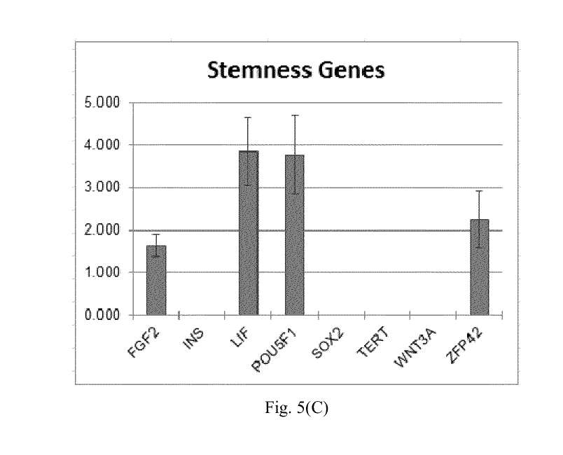

FIG. 5 is a graph of the expression of MSC associated genes. MSC associated genes are genes that are generally found in all mesenchymal stem cells to some degree. FIG. 6 is a graph of the expression of MSC specific genes. These genes are generally unique to mesenchymal stem cells. FIG. 7 is a graph of the expression of Sternness genes. These genes generally are found in cells with more differentiation potential such as embryonic stem cells

These results uniquely demonstrate a resident stem cell population within depots of brown adipose tissue from adult human mediastinum. Cells from this tissue exhibit multi-lineage potential with capacities to undergo osteogenesis, chondrogenesis and both brown and white adipogenesis. Directionally differentiated brown adipocytes exhibit a distinct morphology and gene expression profile, with functional properties characteristic of brown adipose tissue in vivo. These brown adipose-derived stem cells may offer a new target to activate and restore energy homeostasis in vivo for the treatment of obesity and related metabolic disorders.

It is to be understood that while the disclosure has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of distinguishing a brown adipose cell from a white adipose cell, the method comprising,
measuring the expression level of one or more genes in an adipose cell, the one or more genes selected from the genes listed in FIG. 4C;
comparing the measured expression levels to a control, and
correlating the expression level of the one or more genes to an identity as a white adipose cell or a brown adipose cell.

2. The method of claim 1, wherein an increase in expression of one or more of the following genes as compared to the control is indicative that the adipose cell is a brown adipose cell: ACACB, ADRB2, FGF10, KLF15, LIPE, NR1H3, CIDEC, ELOVL3, INHBB, PPARGC1A, and UCP1.

3. The method of claim 1, wherein an increase in expression of LEP as compared to the control is indicative that the adipose cell is a white adipose cell.

4. The method of any one of claims 1-3, comprising measuring the expression level by quantifying transcript levels.

5. The method of claim 1 or 2, comprising measuring the levels of at least two, at least three, at least four, at least five, at least 6, at least 7, at least 8, at least 9, at least 10, or at least 11 genes.

6. The method of claim 2, comprising measuring the levels of any one of ELOVL3, INHBB, PPARGC1A, or UCP 1.

7. The method of claim 2, comprising measuring the levels of any two of ELOVL3, INHBB, PPARGC1A, or UCP 1.

8. The method of claim 2, comprising measuring the levels of any three of ELOVL3, INHBB, PPARGC1A, or UCP 1.

9. The method of claim 2, comprising measuring the levels of ELOVL3, INHBB, PPARGC1A, and UCP1.

10. A method of differentiating an adipose stem cell, the method comprising:
inducing differentiation of an adipose stem cell in vitro;
distinguishing the differentiated stem cell according to the method of claim 1.

11. The method of claim 10, wherein the inducing is performed by contacting the adipose stem cell with a brown adipose cell differentiation media.

12. The method of claim 10, wherein the inducing is performed by contacting the adipose stem cell with FNDC5.
